SEQUENCE LISTING (24 sequences)

SEQ ID NO: 1 - DNA, 2249 bp, Aspergillus sojae
  Aspergillus sojae Ku70
atggctgacgaggatcaatatcgcggagacgaccagatcgatgaggaagaggaggagatcgacgagagtgtacacactttcaaacacacctgaaagcttcggaggctaacatgttatcaaccaaaatagggatacaaaacagtgaaagatgccgttctttttgctatcgaagtcagcgattcgatgctcacccctcgtccatcttccgattcaaagaaacctgcggaggagtcccccacaacggcggcactaaaatgcgcatatcatctcatgcaacaacgcattatctctaatccccgtgacatgatcggtgtgctattatatgggacgcaggcgtccaaattttatgacgaggatgaaaacagtcgaggagacctttcatacccacactgctacctgttcacagacctggatgttccctctgcgcaagaagtcaagaatcttcgggcactggcacaagacggcgatgaatcagaggatgtacttaaggcgtcaggcgagcgggtctcaatggcgaacgtactcttttgcgccaatcaaatattcacgtcaaaagcccccaacttcttgtctcggcgattgttcatagtcaccgataatgatgaccctcatggcgataacaaaagcttgagatccgctgcaactgtacgcgcgaaggacttatacgacctcggtgtcactattgagctgtttccgatttctcggccagaccatgagttcgataccgccaggttctatgacgtaagattatattgactcaatgtgaagtatcgctgctaacagcaattaggatatcatctacaaggcctctccttcggatccagatgccccagcgtacctgcaaactgattccaaggcttctccagctaccggggatgggatatcactgctcagtaccctcctgtccagtatcaattcgagatctgtcccacggcgtgcgcagttctccaacataccattggagctgggaccaaacttcaaaatatctgtctcgggatatcttttgttcaagcgtcaagcacctgccagaaactccttcatctggctcggcggtgaacagccccagattgtcaaaggagtgaccactcaaatcgctgacgacacggctcgcacgattgagaagtgggaaatcaagaaagcttataagtttgggggtgatcaggttgctttcacgcccgaagagatgaagtcactgaggaacttcggtgatcctgtcatccgtataattgggttcaagcccctttctgcacttccgttctgggccaatatcaaacacccttccttcatatacccatcggaagaagatttcgtgggctccacgcgagtcttttctgctttgcatcagacacttctccgggataaaaaggccgcacttgtctggttcattgcgcgtaaaaacgcaagccctgttctgggggctatggtcgctggcgaagagaaactagacgagagtggcgtccagaagtttcctccaggaatgtggataatacctctcccgttcgctgatgacgtccgtcaaaaccctgaaaccacactccatgttgcacccgagccattgatcgatcaaatgcggtatattgttcagcaactgcaacttccaaaggcgtcttacgaccccttcaagtaccctaatccatgtaagcttctgccaacttcctgcacagaaactctggcattaacctattgctctgttagccctccaatggcattatcgcattctacaagccttagcgttagatgaggacctcccggagaagccagaagacaaaacgttgcccagatatcggcagattgataaagtatatcacacattcctattctttccacggatcttgctgaccttcgcttagcgcactggcgactatgtattgtcttgggccgacgagttggaaaagcaatacgcgaaaatatcggcacatggcccgaagagcacactcgtcgaacgaagcgccaaagaccgaacatctgaagttgaggatgcagccccgaagccatacaagaaagtgaaggtggagacagacgagcaaggtgttgaagatgtagtgcgagccccttaccaaaagggatcgctatcgaaggtgactattacctgccccctaggctttaatttggactaactaacgcgcgtgacttgtgtgtagcttactgtacccgtcctcaaaaacttcctgaaagcccatggacgctccgctgctgggaagaaaaaagaactcgttgagcgtgtggaggagtacctggagcagaagtga

SEQ ID NO: 2 - DNA, 2707 bp, Aspergillus sojae
  Aspergillus sojae Ku80 Long
atggcggacaaggaagcaactgtgtatattgtggacgttgggaggtccatgggagaatgtcgcaatggccgatcagtgactgatcttgaatgggccatgcagtatgtctgggatcgcattacaggaacagtgagtggcagtcgtcacaattgggctgcattcgttaaatatcttgctcaatttcagaccaggtggccactggccgtaaaactgccacgatgggtgtgatcggactcaggacagatggtatgtatacttctgaatactgtatgcggttcatacactgaaccaaaaaaattagaaacgtccaacgaacttgaagatgacgtacatttctctcacattgcagttctgtcgaacatcaaacagtatgctttccactctatgataatttggttcgtgcgccaaactgacgaggacgtcaaggtttcttatgccggacattcggaaactggaagatgaattgaaaccgagcaaaacggacaagggagacggtaagttttttgtaagccactaggacctactgtccacttactaaacttcattctctagctatttccgctattatcttggctattcagatgattatcacgcattgcaagaagttgaagtacaggcgcaagatcgccctagtcactaacggacaggggcgcatgagtgatgaggacctgggcgagattgtgaaaaaggtcaaggaagataacatcgagcttgttgttatgtcagtgatttgctacaagatagcaacgaaacaaaaagctaacgtcaagcaggggaattgatttcgatgaccctgagtacggttacaaagaagaagacaaagaccctcgcaaggtagcgatatctcttgcgcagctttattcgtatctaataactaaaacaggccgaaaacgaaactctcttgcgtaccctcgtggaagattgtgatggagtttatggaacattcgagcaggctgtggctgaactagacattccccgtgtcaagtctgtcaggtcagtggcaagctttaaaggatatctccaactaggcaacccagaggattatgactctgctctccgcattcctgttgaaaggtactaccggacttacccggccaaacccccaaccgcaagttctttcgtcctgcgctcagagcctgaagctggacaagaagaggcagagtcatctgaggctgctgctgctacacaaaaagggagccagtctggagatatcggactcactactgtgagaaccatgagaacatatcaagttgaggacaaaagtgcaccgggtgggaaaatcgacatcgaacgagatgacctcgccaaaggatatgagtatggacggacagcagttcacattagtgaaaccgacgagaacatcacgattctcgatacattcgcagggctggagttgatgggcttcatccagactgaccgggtatgtcttgctgaagtcgcctcggtgcatgctctgacacgattaattatagtatcaacgttatatgcacatgtccaacacaaacatcataattgcacaacgtgccaacgacaaagcagcccttgccctttcatcctttatacacgcgctttttgagctagaatgctatgctgttgctcgcctagtcgtgaaagagaacaagccacctgttatagtcttgctcgcgccctcgatcgagcctgattatgaatgccttctcgaagtccagttaccattcgcggaagatgtccgaacctatcggttccctcctctggataaagtgattactgtttctggaaaggttgtgacgcaacaccggaatcttcccaatgatgatttactcgatgtgatgggcaagtacgtgaatagtatggagcttgtcgacgcagatgaggatgggtaggtttatgcctaaaagattccgaatctcttctcattgacaaaaccagggatccagttgagactttccctatcgacgactcgtattcgcctgttttgcaccggattgacgccgccatccgtgctcgggctatacaccctgaccagcccatacctcctccatcagagagactgacaaaattctcacacccacgagaggatctcatcgagagatcacagaaatacctagagaagttgatcgagatagccgatgttaagaagggttggacatcatcccacaatcaagtctatcaggctgctaattctgttaccagttcctcccaaagcgaagggtcgtaagcgcactcgcgaaactgagaagccgctttccggactcgacgtcgatgccctgcttcatcatgaaaagcgcgccaagatatctcccaacaatgccattcccgagttcaagcagactctcgcacaggccgagaatatcgaggccatcaaagacgctacaaagcagatgatggttatcgttgaagatcaaatcaaacacagtctcggtgatgctaactacgaccgggtcattgaagcgctgggcacgatgcgtgacgagttggtatcatacgaagagcctacctcctacaataacttcctaggccagctcaaggataagttgttacaggagaagcttggaggagatcgacaagagctgtggtggcttattcgacgaaacaagctgggacttgtcactcagcgcgagtcggatcaatctagggttaccgatacggaagccaaagaagtaagtctcattaagatgaaggagtgacctagactaaccgcagtttacagttcatgtccgccaaatga

SEQ ID NO: 3 - DNA, 2666 bp, Aspergillus sojae
  Aspergillus sojae Ku80 Short
atggcggacaaggaagcaactgtgtatattgtggacgttgggaggtccatgggagaatgtcgcaatggccgatcagtgactgatcttgaatgggccatgcagtatgtctgggatcgcattacaggaacagtgagtggcagtcgtcacaattgggctgcattcgttaaatatcttgctcaatttcagaccaggtggccactggccgtaaaactgccacgatgggtgtgatcggactcaggacagatggtatgtatacttctgaatactgtatgcggttcatacactgaaccaaaaaaattagaaacgtccaacgaacttgaagatgacgtacatttctctcacattgcagttctgtcgaacatcaaacagtatgctttccactctatgataatttggttcgtgcgccaaactgacgaggacgtcaaggtttcttatgccggacattcggaaactggaagatgaattgaaaccgagcaaaacggacaagggagacggtaagttttttgtaagccactaggacctactgtccacttactaaacttcattctctagctatttccgctattatcttggctattcagatgattatcacgcattgcaagaagttgaagtacaggcgcaagatcgccctagtcactaacggacaggggcgcatgagtgatgaggacctgggcgagattgtgaaaaaggtcaaggaagataacatcgagcttgttgttatgtcagtgatttgctacaagatagcaacgaaacaaaaagctaacgtcaagcaggggaattgatttcgatgaccctgagtacggttacaaagaagaagacaaagaccctcgcaaggtagcgatatctcttgcgcagctttattcgtatctaataactaaaacaggccgaaaacgaaactctcttgcgtaccctcgtggaagattgtgatggagtttatggaacattcgagcaggctgtggctgaactagacattccccgtgtcaagtctgtcaggtcagtggcaagctttaaaggatatctccaactaggcaacccagaggattatgactctgctctccgcattcctgttgaaaggtactaccggacttacccggccaaacccccaaccgcaagttctttcgtcctgcgctcagagcctgaagctggacaagaagaggcagagtcatctgaggctgctgctgctacacaaaaagggagccagtctggagatatcggactcactactgtgagaaccatgagaacatatcaagttgaggacaaaagtgcaccgggtgggaaaatcgacatcgaacgagatgacctcgccaaaggatatgagtatggacggacagcagttcacattagtgaaaccgacgagaacatcacgattctcgatacattcgcagggctggagttgatgggcttcatccagactgaccgggtatgtcttgctgaagtcgcctcggtgcatgctctgacacgattaattatagtatcaacgttatatgcacatgtccaacacaaacatcataattgcacaacgtgccaacgacaaagcagcccttgccctttcatcctttatacacgcgctttttgagctagaatgctatgctgttgctcgcctagtcgtgaaagagaacaagccacctgttatagtcttgctcgcgccctcgatcgagcctgattatgaatgccttctcgaagtccagttaccattcgcggaagatgtccgaacctatcggttccctcctctggataaagtgattactgtttctggaaaggttgtgacgcaacaccggaatcttcccaatgatgatttactcgatgtgatgggcaagtacgtgaatagtatggagcttgtcgacgcagatgaggatgggtaggtttatgcctaaaagattccgaatctcttctcattgacaaaaccagggatccagttgagactttccctatcgacgactcgtattcgcctgttttgcaccggattgacgccgccatccgtgctcgggctatacaccctgaccagcccatacctcctccatcagagagactgacaaaattctcacacccacgagaggatctcatcgagagatcacagaaatacctagagaagttgatcgagatagccgatgttaagaagggttggacatcatcccacaatcaagtctatcaggctgctaattctgttaccagttcctcccaaagcgaagggtcgtaagcgcactcgcgaaactgagaagccgctttccggactcgacgtcgatgccctgcttcatcatgaaaagcgcgccaagatatctcccaacaatgccattcccgagttcaagcagactctcgcacaggccgagaatatcgaggccatcaaagacgctacaaagcagatgatggttatcgttgaagatcaaatcaaacacagtctcggtgatgctaactacgaccgggtcattgaagcgctgggcacgatgcgtgacgagttggtatcatacgaagagcctacctcctacaataacttcctaggccagctcaaggataagttgttacaggagaagcttggaggagatcgacaagagctgtggtggcttattcgacgaaacaagctgggacttgtcactcagcgcgagtcggatcaatctagggttaccgatacggaagccaaagaagtaagtctcattaagatgaaggagtga

SEQ ID NO: 4 - DNA, 2249 bp, Aspergillus oryzae
  Aspergillus oryzae Ku70
atggctgacgaggatcaatatcgtggagacgaccagatcgatgaggaagaggaggagaccgacgagagtgtacacactttcaaacacacctgaaagcttcggaggctaacatgttatcaaccaaaatagggatacaaaacagtgaaagatgccgttctttttgctatcgaagtcagcgattcgatgctcacgcctcgtccatcttccgattcaaagaaacctgcggaggagtcccccacaacggccgcactaaaatgcgcatattatctcatgcaacaacgcattatctctaatccccgtgacatgatcggtgtgctattatatgggacgcaggcgtccaaattttatgacgaggatgaaaatagtcgaggagatctttcatacccacactgctaccttttcacagaccttgatgtcccctctgcgcaagaagtcaagaatcttcgggcactagcacaagacggcgatgaatcaaaggatgtacttaaggcgtcaggcgagcgggtctcaatggcgaacgtactcttttgcgccaatcaaatattcacgtcgaaagcccctaacttcttgtctcggcgattgtttatagtcaccgataatgatgaccctcatggcgataataaaagcttgagatccgcttcaactgtacgcgcgaaggacttatatgacctcggtgtcactattgagctgtttccgatttctcggccaggccatgagttcgataccgccagattctatgacgtaagattatattgactcaatgtgaagtatcgctgctaacagcaattaggatatcatctacaaggcctctccttcggatccagatgccccggcatacctgcaaaccgattccaaggcttctccagccaccggggatgggatatcactgctcaataccctcctgtccaatatcaattcaagatctgtcccacggcgtgcacagttctccaatataccattggagcttggaccaaacttaaaaatatctgtctcaggatatcttttgttcaagcgtcaagcacccgccagaaactccttcatctggctcggcggtgaacagccccagattgtcaaaggagtgaccactcaaatcgctgacgacacggctcgcacgattgaaaagtgggaaattaagaaagcttataagtttggcggtgatcaggttgctttcacgcccgaagagatgaagtcactgaggaacttcggtgatcctgtcatccgtataatagggttcaagcccctctctgcacttccgttctgggccaatatcaaacacccctcctttatatacccatcggaagaagattttgtgggctccacgcgggttttttctgctttgcatcagacactcctccgggataaaaaggccgcacttgtctggttcattgctcgtaaaaatgcaagtcctgttctgggggctatggtcgccggagaagagaaactagacgagagtggcgtccagaagtttcctccaggaatgtggataatacctctcccgttcgctgatgacgtccgtcaaaaccctgaaaccacactccatgttgcacctgagccattgatcgatcaaatgcggtatattgtccagcaattgcaacttccaaaggcgtcttacgacccctttaagtaccctaatccatgtaagcttctgccaacttcctgcacagaaactctggcattaacctattgctctgttagccctccaatggcattatcgcattctacaagccttggcgttggatgaggacctcccggagaagccagaagacaaaacgttgcccagatatcggcagatcgataaagtatatcacacattcctattctttccacggatcttgctgaccttcgcttagcgcactggcgactatgtattgtcttgggccgacgagttggaaaagcaatacgcgaaaatatcggcacatggcccgaagagcacactcgtcaaacgaagcgccaaagaccgaacatctgaagtcgaggatgcagcccagaagccatacaagaaagtgaaggtggagacagacgagcaaggcgttgaagatgtagtgcgagcccattaccagaagggatcgctatcgaaggtgactattacctgccccctaggctttaatttggactaactaacgcgcgtgacttgtgtgtagcttacggtacctgtcctcaaagactttctgaatgcccatggacgctccgctgctgggaagaaagctgatctcgttgagcgtgtggaggagtatttggagcagaaatga

SEQ ID NO: 5 - DNA, 2703 bp, Aspergillus oryzae
  Aspergillus oryzae Ku80 Long
atggcggacaaggaagcaactgtgtatattgtggacgttgggaggtccatgggagaatgtcgcaatggccgatcagtgactgatcttgaatgggccatgcagtatgtctgggatcgcattacaggaacagtgagtggcagtcgtcacaattggaccgtattcgttaaataccttgctcaatttcaaaccaggtggccactggccgcaaaactgccatgatgggtgtgattggactcaggacagatggtatgtatacttctgaatactgtatgcggttcatacgctgatccaaaaattagaaacgtccaacgaacttgaagatgacgtacatttctctcacattgcagttctgtcgaacctcaaacagtatgctttccactctatgataatttggtttgtgcgccaaactgacgaggacgtcaaggtttcttatgccggacattcggaaactggaagatgaactgaaaccgagcaaaacggacaaaggagacggtaagctttttgagagccactaggacctactgtccaatttactaaactttgttctctagctatttccgctattatcttggctattcagatgattatcacgcattgcaagaagttgaagtacaggcgcaagatcgtcctcgtcactaacggacaggggcgcatgagcgatgaagacctgggcgagattgtgaagaaggtcaaggaagataacatcgagcttgttgttatgtcagtgatttgctaccagatagcaacgaaacaaaagctaacttcaagcaggggaattgatttcgatgaccctgagtacggttacaaagaagaagacaaagaccctcacaaggtagcgatatctctcgcgcagctttgttcttttctaacaactaaaacaggccgaaaatgaaactctcttgcgtacccttgtggaagattgtgatggagtctatggaacattcgagcaggctgtggctgaactagacatcccccgtgtcaagtctgtcaggtcagtggcaagcttcaaaggatatctccaactaggcaacccagaggagtatgactctgctctccgcattcctgttgaaaggtactatcggacttacccggccaaacccccgaccgcaagttctttcgtcctgcgctcagagcctgaagctggacaagaagaggcagagtcatctgaggctgctgctgctacgcaaaaagggagccaatctggagatgccggattgaccactgtgagaaccatgagaacatatcaagttgaggacaaaagtgcaccgggtgggaaaatcgacatcgaacgagatgagctcgccaaaggatatgagtatggacggacagcagttcacattagtgaaactgacgagaacatcacgattctcgatacattcgcagggctggagttgatgggcttcatccagactgaccaggtatgtcttgctgaagtcgcctcggtgcatgctctgacacacgattatagtatcaacgttatatgcacatgtccaacacaaacatcataattgcacaacgtgccaacgacaaagcagctcttgccctttcatcctttatacacgccctttttgagctagaatgctatgctgttgctcgcctagttgtgaaagagaacaagccaccagttatagtcttgctcgcgccctcgatcgagcctgagtatgaatgccttctcgaagtccagttaccatttgcggaagatgtccgaacctatcggttccctcctctggataaagtgattactgtttccggaaaggttgtgacacaacaccggaatcttcccagtgatgatttactcgatgtgatgggcaagtacgtgaatagtatggagcttgtcgacgcagatgaggatgggtaggtttatgcctaaaagattccgaatatcttctcattgacataaccagggatccagtggagactttccctatcgacgactcgtattccccagttttgcaccggattgacgccgccatccgtgctcgggctatacatcctgaccagcccatacctcctccatcagagagactgacaaaattctcacacccacgagaggatctcatcgagaaatcacagaaacacctagagaagttgatcgagatagccgatgttaagaagggttggacatcaccccacaatcaagtctgtcagactgctaattcagttaccagttcctcccaaagcgaagggtcgcaagcgcactcgtgaaaccgaaaagccactttccggactcgacgtcgatgccctgcttcatcatgaaaagcgcgtcaagatatctcccaacaatgccattcctgagttcaagcagactctcgcacaggccgagaatatcgaggccatcaaagacgctacaaagcagatgatggtcatcgttgaagatcaaatcaaacacagtctcggtaatgctaactacgaccgggtcattgaagcgctgggcacgatgcgtgacgagttggtatcttacgaagagcctgcctcctacaatgacttcctgggccagctcaaggataagttactgcaggagaagcttggaggagaccgacaagagctgtggtggcttgttcgacgaaacaagctgggacttgtcactcagcgcgagtcggatcaatctagggttaccgatacggaagccaaagaagtaagtctcactaagatgaaggagtgacctagactaactgcagtttacagttcatgtccgccagatga

SEQ ID NO: 6 - DNA, 2662 bp, Aspergillus oryzae
  Aspergillus oryzae Ku80 Short
atggcggacaaggaagcaactgtgtatattgtggacgttgggaggtccatgggagaatgtcgcaatggccgatcagtgactgatcttgaatgggccatgcagtatgtctgggatcgcattacaggaacagtgagtggcagtcgtcacaattggaccgtattcgttaaataccttgctcaatttcaaaccaggtggccactggccgcaaaactgccatgatgggtgtgattggactcaggacagatggtatgtatacttctgaatactgtatgcggttcatacgctgatccaaaaattagaaacgtccaacgaacttgaagatgacgtacatttctctcacattgcagttctgtcgaacctcaaacagtatgctttccactctatgataatttggtttgtgcgccaaactgacgaggacgtcaaggtttcttatgccggacattcggaaactggaagatgaactgaaaccgagcaaaacggacaaaggagacggtaagctttttgagagccactaggacctactgtccaatttactaaactttgttctctagctatttccgctattatcttggctattcagatgattatcacgcattgcaagaagttgaagtacaggcgcaagatcgtcctcgtcactaacggacaggggcgcatgagcgatgaagacctgggcgagattgtgaagaaggtcaaggaagataacatcgagcttgttgttatgtcagtgatttgctaccagatagcaacgaaacaaaagctaacttcaagcaggggaattgatttcgatgaccctgagtacggttacaaagaagaagacaaagaccctcacaaggtagcgatatctctcgcgcagctttgttcttttctaacaactaaaacaggccgaaaatgaaactctcttgcgtacccttgtggaagattgtgatggagtctatggaacattcgagcaggctgtggctgaactagacatcccccgtgtcaagtctgtcaggtcagtggcaagcttcaaaggatatctccaactaggcaacccagaggagtatgactctgctctccgcattcctgttgaaaggtactatcggacttacccggccaaacccccgaccgcaagttctttcgtcctgcgctcagagcctgaagctggacaagaagaggcagagtcatctgaggctgctgctgctacgcaaaaagggagccaatctggagatgccggattgaccactgtgagaaccatgagaacatatcaagttgaggacaaaagtgcaccgggtgggaaaatcgacatcgaacgagatgagctcgccaaaggatatgagtatggacggacagcagttcacattagtgaaactgacgagaacatcacgattctcgatacattcgcagggctggagttgatgggcttcatccagactgaccaggtatgtcttgctgaagtcgcctcggtgcatgctctgacacacgattatagtatcaacgttatatgcacatgtccaacacaaacatcataattgcacaacgtgccaacgacaaagcagctcttgccctttcatcctttatacacgccctttttgagctagaatgctatgctgttgctcgcctagttgtgaaagagaacaagccaccagttatagtcttgctcgcgccctcgatcgagcctgagtatgaatgccttctcgaagtccagttaccatttgcggaagatgtccgaacctatcggttccctcctctggataaagtgattactgtttccggaaaggttgtgacacaacaccggaatcttcccagtgatgatttactcgatgtgatgggcaagtacgtgaatagtatggagcttgtcgacgcagatgaggatgggtaggtttatgcctaaaagattccgaatatcttctcattgacataaccagggatccagtggagactttccctatcgacgactcgtattccccagttttgcaccggattgacgccgccatccgtgctcgggctatacatcctgaccagcccatacctcctccatcagagagactgacaaaattctcacacccacgagaggatctcatcgagaaatcacagaaacacctagagaagttgatcgagatagccgatgttaagaagggttggacatcaccccacaatcaagtctgtcagactgctaattcagttaccagttcctcccaaagcgaagggtcgcaagcgcactcgtgaaaccgaaaagccactttccggactcgacgtcgatgccctgcttcatcatgaaaagcgcgtcaagatatctcccaacaatgccattcctgagttcaagcagactctcgcacaggccgagaatatcgaggccatcaaagacgctacaaagcagatgatggtcatcgttgaagatcaaatcaaacacagtctcggtaatgctaactacgaccgggtcattgaagcgctgggcacgatgcgtgacgagttggtatcttacgaagagcctgcctcctacaatgacttcctgggccagctcaaggataagttactgcaggagaagcttggaggagaccgacaagagctgtggtggcttgttcgacgaaacaagctgggacttgtcactcagcgcgagtcggatcaatctagggttaccgatacggaagccaaagaagtaagtctcactaagatgaaggagtga

SEQ ID NO: 7 - DNA, 27 bp, Artificial Sequence
  Synthetic primer
caatcttcgtgcacataagttcacgtt

SEQ ID NO: 8 - DNA, 44 bp, Artificial Sequence
  Synthetic primer
gttgaacatatctgacaagccttccgagcagacggtgctaatca

SEQ ID NO: 9 - DNA, 44 bp, Artificial Sequence
  Synthetic primer
tgactcgttattctttgctcatcgcgtcgtggcgaattctctcg

SEQ ID NO: 10 - DNA, 27 bp, Artificial Sequence
  Synthetic primer
ggcggtgcgctggcggaaaaacgcaga

SEQ ID NO: 11 - DNA, 23 bp, Artificial Sequence
  Synthetic primer
tccccaccacactgtaattcatc

SEQ ID NO: 12 - DNA, 44 bp, Artificial Sequence
  Synthetic primer
tgactcgttattctttgctcatagcagcttcggttgtctacttt

SEQ ID NO: 13 - DNA, 23 bp, Artificial Sequence
  Synthetic primer
tccgcattggcttaacccagagt

SEQ ID NO: 14 - DNA, 44 bp, Artificial Sequence
  Synthetic primer
tgactcgttattctttgctcatagccgggatttgtaccattggt

SEQ ID NO: 15 - DNA, 27 bp, Artificial Sequence
  Synthetic primer
acggcttcggcgttacctcgttcaacc

SEQ ID NO: 16 - DNA, 27 bp, Artificial Sequence
  Synthetic primer
cccccacatgccgttgacctggtcctc

SEQ ID NO: 17 - DNA, 26 bp, Artificial Sequence
  Synthetic primer
gtagcaatggtcatataaacgagcga

SEQ ID NO: 18 - DNA, 26 bp, Artificial Sequence
  Synthetic primer
tatactccgctctaacgtgctgtgtg

SEQ ID NO: 19 - DNA, 23 bp, Artificial Sequence
  Synthetic primer
aggcttgtcagatatgttcaacg

SEQ ID NO: 20 - DNA, 23 bp, Artificial Sequence
  Synthetic primer
atgagcaaagaataacgagtcaa

SEQ ID NO: 21 - DNA, 30 bp, Artificial Sequence
  Synthetic primer
ggtgcactgcgtgtcgtcctgcagactaca

SEQ ID NO: 22 - DNA, 30 bp, Artificial Sequence
  Synthetic primer
gtcgccgccggatctcattgcaaaagctct

SEQ ID NO: 23 - DNA, 27 bp, Artificial Sequence
  Synthetic primer
gcacaccggacggaattgaaatatctc

SEQ ID NO: 24 - DNA, 27 bp, Artificial Sequence
  Synthetic primer
ggcggtgcgctggcggaaaaacgcaga

(19) United States
(12) Patent Application Publication
(10) Pub. No.: US 2009/0035863 A1
(43) Pub. Date: Feb. 5, 2009

(54) Method for the deletion of a large chromosomal region

(75) Inventors: Tadashi Takahashi, Noda-shi (JP); Yasuji Koyama, Noda-shi (JP)

Correspondence Address: BIRCH STEWART KOLASCH & BIRCH, PO BOX 747, FALLS CHURCH, VA, 22040-0747, US

(73) Assignee: NODA INSTITUTE FOR SCIENTIFIC RESEARCH, Noda-shi (JP)

(21) Appl. No.: 12/081,999

(22) Filed: Apr. 24, 2008

(30) Foreign Application Priority Data
Apr. 25, 2007 (JP) 2007-115013

(51) Int. Cl. C12N 15/80; C12N 1/15

(52) U.S. Cl. 435/471; 435/254.11

(57) ABSTRACT

In order to efficiently delete a large chromosomal region with a length of from several tens to hundreds kb such as a cluster of biosynthesis genes encoding a toxic substance such as Aflatoxin, the present invention provides a method for the deletion of a large chromosomal region comprising transforming a transformant having an increased frequency of homologous recombination due to suppression of a Ku gene, which is a mitosporic filamentous fungus belonging to Trichocomaceae with a vector for the deletion of a chromosomal region, which comprises a pair of homologous region arms having a nucleotide sequence that is homologous with a fragment at either end of said chromosomal region, and deleting the chromosomal region by means of homologous recombination between the resulting transformant and said vector.

### TECHNICAL FIELD

The present invention relates to a method for the deletion of a large chromosomal region comprising transforming a transformant having an increased frequency of homologous recombination due to suppression of a Ku gene, which is a mitosporic filamentous fungus belonging to Trichocomaceae, for example, Aspergillus such as Aspergillus sojae and Aspergillus oryzae with a vector for the deletion of a chromosomal region, and deleting the chromosomal region by means of homologous recombination between the resulting transformant and said vector.

### BACKGROUND OF THE INVENTION

Aspergillus strains such as Aspergillus sojae and Aspergillus oryzae have been industrially used in the production of brewed food such as soy sauce, sake (rice wine), soybean paste, etc. With a recent determination of the whole genomic sequence of Aspergillus oryzae and development of an exhaustive analysis of gene expression using a micro-array, it has been expected that genetic modification of their genes, especially their chromosomal modification would increase the productivity of an enzyme and improve a growth rate of these filamentous fungi.

However, as Aspergillus strains have a very low frequency of homologous recombination, it has been very difficult to produce a strain with a large area-deletion of an arbitrary chromosomal region by conventional methods. For example, as the frequency of homologous recombination is as low as 1˜2%, it will be necessary to prepare several tens to hundreds of transformants and select a desired strain from them in order to integrate even a single vector into an optional area of its chromosome (Non-Patent Document 1). Furthermore, it is necessary to select a recombinant strain having a recombination between both ends of an area to be deleted for obtaining the strain with a large-area deletion. But, it would be very difficult to select such recombinant for Aspergillus strains that have the very low frequency of homologous recombination. However, a recent study by the present inventors has revealed that the disruption of a gene involved in non-homologous recombination will significantly increase the frequency of gene targeting (homologous recombination) (Patent Document 1).

By the way, Aflatoxin is a toxin produced by fungi belonging to Aspergillus such as Aspergillus flavus or Aspergillus parasiticus, and is known as the strongest cancer-causing agent among natural substances. There have been big problems about contamination of cereal due to these molds in tropical region. There was death en mass of turkeys in England in 1960, which was caused by uptake of the cereal contaminated by the mold (an accident of turkey's X disease). Since the above Aspergillus strains are closely-related to Aflatoxin-producing strains, they have been studied in a wide variety of views with suspicion of their Aflatoxin-producing capability (Non-Patent Document 2). As a result, it has been confirmed that although they will not produce Aflatoxin under any conditions, they have a homologue of a gene cluster of about 60 Kb for the Aflatoxin-biosynthesis of comprised the Aflatoxin-producing strains (Non-Patent Document 3).

It has been therefore strongly desired that the gene cluster should be removed from the Aspergillus strains for a further increase of safety of foods. However, it has been very difficult to prepare a mutant strain having deletion of the above Aflatoxin cluster since a set of genes in this cluster are not expressed (Non-Patent Document 4), making it impossible to carry out selection based on its phenotype in addition to the low frequency of their homologous recombination.

### [Patent Document 1] JP 2006-158269

[Non-Patent Document 1] Takahashi et al., Mol. Gen. Genet. (2004) 272:344-52

### [Non-Patent Document 2] Matsushima et al., Journal of the Japan Soy Sauce Research Institute, (2004) 31:5-11

### [Non-Patent Document 3] Yu et al. (2004) Appl Environ Microbiol 70:1253-1262

[Non-Patent Document 4] Matsushima et al., Appl Microbiol Biothechnol 55: 771-776

### DISCLOSURE OF THE INVENTION

### Problems to be Solved by the Present Invention

The main purpose of the present invention is therefore to provide a method for efficiently deleting a large chromosomal region with a length of from several tens to hundreds kb such as a cluster of biosynthesis genes encoding a toxic substance such as Aflatoxin, and to a transformant produced by said method, which will not produce the toxic substance even after being manipulated with genetic engineering.

The present inventors succeeded in efficiently producing a strain having the deletion of such a large region as from ten-odd kb to about 200 kb by using such Aspergillus strains with an increased frequency of homologous recombination as disclosed in Patent Document 1, and have completed the present invention.

Thus, the present invention is related to a method for the deletion of a large chromosomal region comprising transforming a transformant having an increased frequency of homologous recombination due to suppression of a Ku gene, which is a mitosporic filamentous fungus belonging to Trichocomaceae, for example, Aspergillus such as Aspergillus sojae and Aspergillus oryzae with a vector for the deletion of a chromosomal region, which comprises a pair of homologous arms having a nucleotide sequence that is homologous with a fragment at either end of said chromosomal region, and deleting the chromosomal region by means of homologous recombination between the resulting transformant and said vector, and to a transformant produced by said method, which has a deleted region in chromosome. An example of the present method is illustrated in FIG. 1.

According to the present invention, it is now possible to efficiently delete a large chromosomal region with a length of from several tens to hundreds kb such as a cluster of Aflatoxin-biosynthesis genes. As a result, it makes it easy to produce a strain having the deletion of a gene cluster occupying a large area in the chromosome, which is very preferable from safety point of view.

### BRIEF DESCRIPTION OF DRAWINGS

FIG. 1 is a schematic figure showing one example of the method according to the present invention.

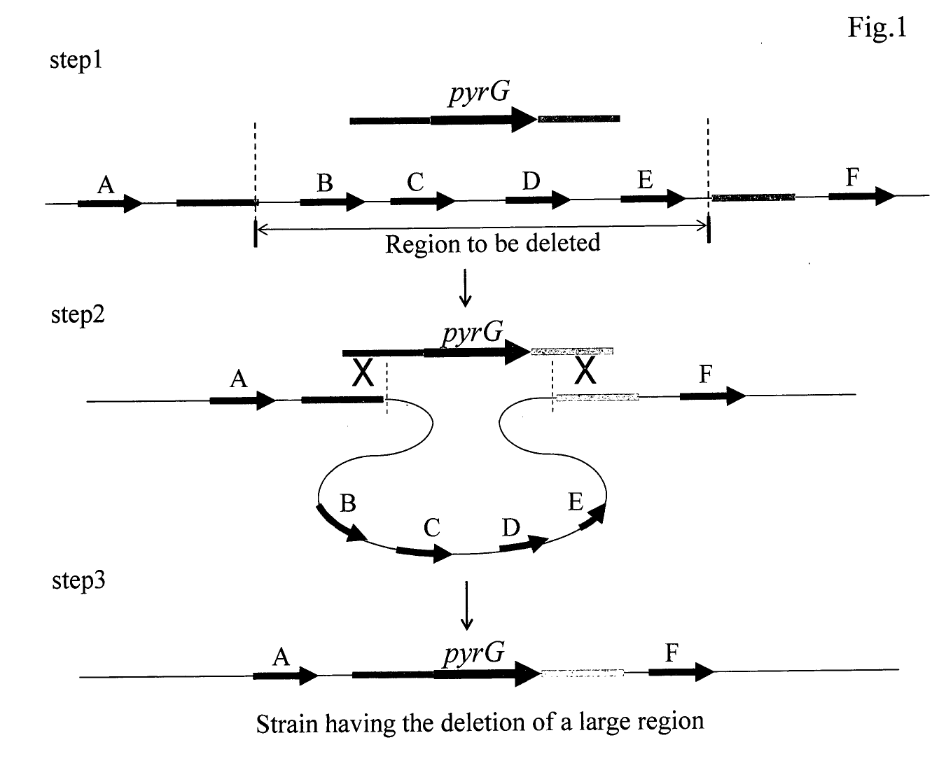

FIG. 2 shows a summary and photographs in electrophoresis showing the results of PCR analysis of the genome of a strain having the deletion of a chromosomal region produced by the method according to present invention using Aspergillus sojae ASKUPTR8 strain.

FIG. 3 shows a summary and photographs in electrophoresis showing the results of Southern hybridization of the genome of a strain having the deletion of a cluster (60 kb) of biosynthesis genes encoding Aflatoxin produced by the method according to present invention using Aspergillus sojae ASKUPTR8 strain.

FIG. 4 shows a summary and photographs in electrophoresis showing the results of Southern hybridization of the genome of a strain having the deletion of a 190 kb region in the chromosome produced by the method according to present invention using Aspergillus sojae ASKUPTR8 strain.

FIG. 5 shows a summary and photographs in electrophoresis showing the results of Southern hybridization of the genome of a strain having the deletion of a 200 kb region in the chromosome produced by the method according to present invention using Aspergillus sojae ASKUPTR8 strain.

FIG. 6 shows a summary and photographs in electrophoresis showing the results of PCR analysis of the genome of a strain having the deletion of a chromosomal region produced by the method according to present invention using RkuN16ptr1 strain.

FIG. 7 shows a summary and photographs in electrophoresis showing the results of Southern hybridization of the genome of a strain having the deletion of a cluster (60 kb) of biosynthesis genes encoding Aflatoxin produced by the method according to present invention using RkuN16ptr1 strain.

### BEST MODE FOR CARRYING OUT THE PRESENT INVENTION

The “transformant having an increased frequency of homologous recombination due to suppression of a Ku gene, which is a mitosporic filamentous fungus belonging to Trichocomaceae” that was used in the present invention may be prepared by the method disclosed in Patent Document 1. Thus, the transformant can be prepared by suppressing the Ku gene of a mitosporic filamentous fungus belonging to Trichocomaceae. The scope and kinds of the fungus that may be mycologically classified into the “mitosporic filamentous fungus belonging to Trichocomaceae” are obvious for those skilled in the art. There may be mentioned as its representative examples strains belonging to Aspergillus such as Aspergillus sojae and Aspergillus oryzae, and strains belonging to penicillium. These strains may be commercially available from public depository authorities such as American Type Culture Collection (ATCC).

As already described in the above, the Ku gene such as Ku70 and Ku80 is a gene involved in the non-homologous recombination mechanism. Its representative examples include Ku70 gene (SEQ ID NO. 1) and Ku80 gene (SEQ ID NO. 2 or SEQ ID NO. 3) derived from Aspergillus sojae, and Ku70 gene (SEQ ID NO. 4) and Ku80 gene (SEQ ID NO. 5 or SEQ ID NO. 6) derived from Aspergillus oryzae. Homology (identity) in an amino acid level between the above Ku70 genes and between the above Ku80 genes was found to be as high as 95% or more. On the other hand, the homology in an amino acid level between these genes and homologues of Neurospora crassa is about 50%.

Accordingly, there may be mentioned as preferable examples of the Ku gene, a gene that encodes a protein consisting of an amino acid sequence represented by any one of SEQ ID Nos 1-6, or a protein consisting of the same amino acid sequence wherein one or several amino acid residues are replaced, deleted, or added, or a gene that encodes a protein having a high homology such as about 90% or more, preferably about 95% or more on a total average to the amino acid sequence represented by any one of SEQ ID Nos 1-6 and having a function relating to non-homologous recombination mechanism.

Furthermore, preferable examples of the Ku gene may include (a) DNA comprising a coding region represented by any one of SEQ ID Nos 1-6, or DNA being hybridized with DNA consisting a base sequence complementary with that of the DNA (a) under stringent conditions, and encoding a protein having the function relating to non-homologous recombination mechanism.

The coding region in any one of SEQ ID Nos 1-6 was determined based on the information about the genomic sequences of Aspergillus sojae and Aspergillus oryzae, comparison with the sequences of their homologues of Neurospora crassa and rules concerning an intron sequence such as GT-AG rule. The genomic sequence of the Ku gene of Aspergillus sojae was determined by amplifying a fragment in PCR using a genomic DNA of Aspergillus sojae ATCC46250 as a template and primers kuU459-KuL4222 and ku2U830-ku2L4937 (prepared based on the genomic sequences of Aspergillus oryzae and their homologues of Neurospora crassa), cloning the resulting fragment by means of TOPO-TA cloning kit (Invitrogen Co.) and subjecting it to a conventional sequence determination.

The hybridization may be performed in accordance with a method known in the art, for example, those described in Molecular cloning third. Ed. Cold Spring Harbor Lab. Press, 2001). When a commercially available library is used, the hybridization may be done according to instructions attached to it.

The hybridization may be performed in accordance with a method known in the art, for example, that described in Current protocols in molecular biology (edited by Frederick M. Ausubel et al., 1987). When a commercially available library is used, the hybridization may be done according to instructions attached to it.

The term “stringent conditions” means in this specification, for example, those of sodium concentration of 150˜900 mM, preferably 600˜900 mM, pH of 6˜8 at 60° C.˜68° C.

The DNA that is hybridized with the DNA consisting of a base sequence complementary with that of the DNA comprising a coding region represented by any one of SEQ ID Nos 1-6 may include, for example, DNA consisting of a base sequence having identity (homology) with the whole base sequence of said DNA, of about 90% or more, preferably of about 95% or more on a total average. The identity between the base sequences may be determined by means of algorithm known to those skilled in the art, such as BLAST.

The suppression of the Ku gene in the transformant according to the present invention may be performed by any method known to those skilled in the art such as those actually described in the examples of the present specification. For example, the Ku gene may be disrupted by means of a Ku gene-disruption vector, or inactivated by means of antisense RNA method using a vector expressing an antisense of the Ku gene. The suppression of the Ku gene may be also attained by various methods based on RNA interference. The resulting transformant may have the frequency of homologous recombination that is increased by at least 10 times, preferably by at least 60 times.

Examples of the above transformants are Aspergillus sojae ASKUPTR8 strain (wh, ΔpyrG, Ku70::ptrA) and Aspergillus oryzae RkuN16ptr1 strain. The Aspergillus sojae ASKUPTR8 strain was deposited at the International Patent Organism Depository of National Institute of Advanced Industrial Science and Technology at AIST Tsukuba Central 6, 1-1, Higashi 1-chome Tsukuba-shi, Ibaraki-ken 305-8566 Japan on Dec. 2, 2004 with Accession No. FERM P-20311, and then transferred to international deposit under Budapest Treaty on Nov. 17, 2005 with Accession No. FERM BP-10453.

The vector used for the deletion of a certain chromosomal region in the method according to the present invention is characterized by comprising a pair of homologous region arms having a nucleotide sequence that is homologous with a fragment at either end of said chromosomal region. The length of the homologous region arms may be optionally selected depending on the chromosomal region to be deled and the like, being preferably in a certain range, for example, about 1-3 kb, in order to obtain a high frequency of homologous recombination. The phrase “nucleotide sequence that is homologous with a fragment at either end of said chromosomal region” means that a nucleotide sequence of said vector used for the deletion of a certain chromosomal region and that in the chromosome of the transformant have such a sufficient identity that homologous recombination will just occur between them. It is therefore not require that their nucleotide sequences are completely the same with each other.

When the chromosomal region to be deleted constitutes a cluster of biosynthesis genes encoding a toxic substance, the homologous region arms in the vector used for the deletion of a certain chromosomal region may have a nucleotide sequence of a gene or a part thereof that is located in the most marginal or peripheral part at either end of the region to be deleted. In such case, said nucleotide sequence may be amplified by any known DNA amplification technique such as PCR, and the homologous region arms may be then easily prepared by using the thus amplified sequence. Accordingly, in the case where the chromosomal region to be deleted is a cluster of Aflatoxin, a nucleotide sequence comprising moxY gene and pksA gene, respectively, may be used as the homologous region arm.

In the above case, as the nucleotide sequence of a gene or a part thereof that is located in the most marginal or peripheral part at both ends of the cluster of genes is comprised in the homologous region arms, said gene or a part thereof may be left even after the deletion of the subject chromosomal region. However, as the other genes in the cluster have been deleted, the toxic substance will never be produced any more. Thus, one of the purposes of the present invention, the provision of a transformant preferable in view of safety, is sufficiently fulfilled. Thus, all of the genes involved in biosynthesis of a substance are not necessarily comprised in the region (the cluster of genes) to be deleted in the method according to the present invention.

Alternatively, as long as the function of the gene that has been left after the deletion of the subject chromosomal region is not maintained any more, such as being incompetent regarding transcription, translation of a functional protein or peptide, due to the loss of a part of said gene, the advantages of the present invention will be attained. Thus, it is not necessary to completely delete the gene located at one of the both ends of the cluster of genes to be deleted.

Furthermore, in order to efficiently select the transformant having the deletion of a certain chromosomal region produced by homologous recombination according to the present method, it is preferable for any suitable selective marker gene known for those skilled in the art to be comprised between the pair of the homologous region arms of the vector for the deletion of a chromosomal region. The selective marker gene includes pyrG, sC, niaD and the like. As a result, said transformant having the deletion of a part of the chromosome, which is produced by the method according to the present invention, may be easily selected by using the above marker during a process of culture.

There is no limitation on size and location in the chromosome, etc. of the chromosomal region to be deleted. It is possible to delete a large chromosomal region with a length of from several tens to hundreds kb, for example 15-200 kb by the present invention. A preferable example of such chromosomal region is the cluster of genes involved in biosynthesis of substances such as Aflatoxin, which should preferably not be produced by the strain from a safety aspect. The size and location in the chromosome of said genes as well as nucleotide sequences located in the most marginal or peripheral part at both ends of the region to be deleted are known for those skilled in the art.

The transformation with the vector for the deletion of a chromosomal region may be carried out by any method known for those skilled in the art, such as protoplast PEG method, electroporation and the like.

The present invention will be specifically explained below with reference to the examples, which should not be construed to limit the scope of the present invention. Unless otherwise described, the means and conditions for gene engineering techniques in the following examples are usual ones known to those skilled in the art, such as those described in Japanese Patent Publication No. 1998-80196.

### EXAMPLE

### Strains

Aspergillus sojae ASKUPTR8 strain (wh, ΔpyrG, Ku70::ptrA) and Aspergillus oryzae RkuN16ptr1 strain (wh, ΔpyrG, Ku70::ptrA) were used. Aspergillus sojae ASKUPTR8 is a Δ Ku70 (Ku70-disruption) strain prepared from Aspergillus sojae I-6 strain, which in turn is a ΔpyrG strain prepared from ATCC46250 (Takahashi et al. 2004. Aspergillus oryzae RkuN16ptr1 strain is a ΔKu70 strain derived from RIB 40 strain according to the method disclosed in Patent Document 1.

### Culture Medium:

Polypeptone dextrin (PD) medium (polypepton 1%, dextrin 2%, KH₂PO₄ 0.5%, NaNO₃ 0.1%, MgSO₄ 0.05%, casamino acid 0.1%, pH 6.0), CzapekDox (CZ) minimum medium, and 1.2M sorbitol CZ (as regeneration medium) were used. CZ medium containing 2 mg/ml 5fluoroortic acid (SIGMA) and 20 mM Uridine was used for positive selection of a pyrG⁻ (Uridine-auxotrophy) strain.

### Transformation:

Conidium was inoculated on liquid PD medium (50 ml) containing 20 mM Uridine in a conical flask (150 ml) and subjected to shake culture for about 20 hours at 30° C., followed by collection of mycelium. The collected mycelium was washed with 0.7M KCl buffer, shaken gently in 0.7M KCl buffer containing 1% Lysing enzyme (Sigma Co.) for 3 hours at 30° C. to prepare protoplast. The protoplast was washed with 1.2M sorbitol buffer, and tranformed by means of a protoplast PEG method. Regeneration of the resulting transformant was carried out on 1.2M sorbitol CZ medium containing 0.5% agar.

### Construction of a Vector for the Deletion of a Large Region

Primers used in the present examples are listed in Table 1 below.

TABLE 1

| | |
|---|---|
| Primers | Sequence (5′ to 3′) |

| | |
|---|---|
| A | CAATCTTCGTGCACATAAGTTCACGTT |
| B | GTTGAACATATCTGACAAGCCTTCCGAGCAGACGGTGCTAAT |
| | CA |
| C | TGACTCGTTATTCTTTGCTCATCGCGTCGTGGCGAATTCTCT |
| | CG |
| D | GGCGGTGCGCTGGCGGAAAAACGCAGA |
| E | TCCCCACCACACTGTAATTCATC |
| F | TGACTCGTTATTCTTTGCTCATAGCAGCTTCGGTTGTCTACT |
| | TT |
| G | TCCGCATTGGCTTAACCCAGAGT |
| H | TGACTCGTTATTCTTTGCTCATAGCCGGGATTTGTACCATTG |
| | GT |
| pksU | ACGGCTTCGGCGTTACCTCGTTCAACC |
| AF-L | CCCCCACATGCCGTTGACCTGGTCCTC |
| 190L | GTAGCAATGGTCATATAAACGAGCGA |
| 200L | TATACTCCGCTCTAACGTGCTGTGTG |
| PU | AGGCTTGTCAGATATGTTCAACG |
| PL | ATGAGCAAAGAATAACGAGTCAA |
| pkU | GGTGCACTGCGTGTCGTCCTGCAGACTACA |
| pkL | GTCGCCGCCGGATCTCATTGCAAAAGCTCT |
| moxU | GCACACCGGACGGAATTGAAATATCTC |
| moxL | GGCGGTGCGCTGGCGGAAAAACGCAGA |

### Construction of a Vector for the Deletion of a Cluster (60 kb) of Biosynthesis Genes Encoding Aflatoxin

A vector (pPkvb1) for the deletion of a cluster of biosynthesis genes encoding Aflatoxin was constructed by means of fusion PCR. It comprised a pair of arms of 2 kb each in both ends having pksA and moxY, respectively, and pyrG as a selective marker. Thus, the arm having pksA, the arm having moxA and the pyrG marker region were amplified by the 1^{st} PCR using the primers A-B, C-D and PU-PL, respectively, and Aspergillus genomic DNA as a template. The resulting amplified fragments were then purified with QIAquick Gel Extraction kit. The 2^{nd} PCR was carried out using the primers pksU-AF-L and the mixture of the above purified fragments as a template to obtain a vector (pPkv1) for the deletion of the cluster of biosynthesis genes encoding Aflatoxin.

### Construction of a Vector for the Deletion of a 190 kb Region

A vector (pPk217-20k) for the deletion of a 190 kb region was constructed by means of fusion PCR. It comprised a pair of arms of 2 kb each in both ends, one of which having pksA and the other being located at 186 kb distance. Thus, the arm having pksA, the other arm and the pyrG marker region were amplified by the 1^{st} PCR using the primers A-B, E-F and PU-PL, respectively, and Aspergillus genomic DNA as a template. The resulting amplified fragments were then purified with QIAquick Gel Extraction kit. The 2^{nd} PCR was carried out using the primers pksU-190L and the mixture of the above purified fragments as a template to obtain a vector (pPk217-20k) for the deletion of the 190 kb region.

### Construction of a Vector for the Deletion of a 200 kb Region

A vector (pPk217) for the deletion of a 200 kb region was constructed by means of fusion PCR. It comprised a pair of arms of 2 kb each in both ends, one of which having pksA and the other being located at 203 kb distance. Thus, the arm having pksA, the other arm and the pyrG marker region were amplified by the 1^{st} PCR using the primers A-B, G-H and PU-PL, respectively, and Aspergillus genomic DNA as a template. The resulting amplified fragments were then purified with QIAquick Gel Extraction kit. The 2^{nd} PCR was carried out using the primers pksU-200L and the mixture of the above-purified fragments as a template to obtain a vector (pPk217) for the deletion of the 200 kb region.

### Conditions of Fusion PCR

The fusion PCR was carried out in accordance with general procedures (Yang et al., (2004) Eukaryot Cell 3: 1359-1362). 1^{st} PCR: keeping for one min and 30 seconds at 94° C., followed by repeating 35 times a cycle of heating for 20 seconds at 94° C., for one second at 70° C., for 30 seconds at 55° C., and for 2 min. at 72° C.; 2^{nd} PCR: keeping for one min and 30 seconds at 94° C., followed by repeating 35 times a cycle of heating for 20 seconds at 94° C., for one second at 70° C., for 30 seconds at 55° C., and for 5 min. and 30 seconds at 72° C. The amplification was done with KOD plus (TOYOBO)

### Southern Hybridization

Southern hybridization was done in accordance with general procedure with a membrane filter of Hybond-N+ (Amersham Pharmacia Co.). Detection was made by means of DIG Luminescent Detection Kit (Roche) in accordance with a method recommended by the manufacturer. Probes for pksA and moxY were prepared by means of PCR DIG Probe Synthesis Kit (Roche) using the primers pkU-pkL and moxU-moxL, respectively.

### Results and Discussion

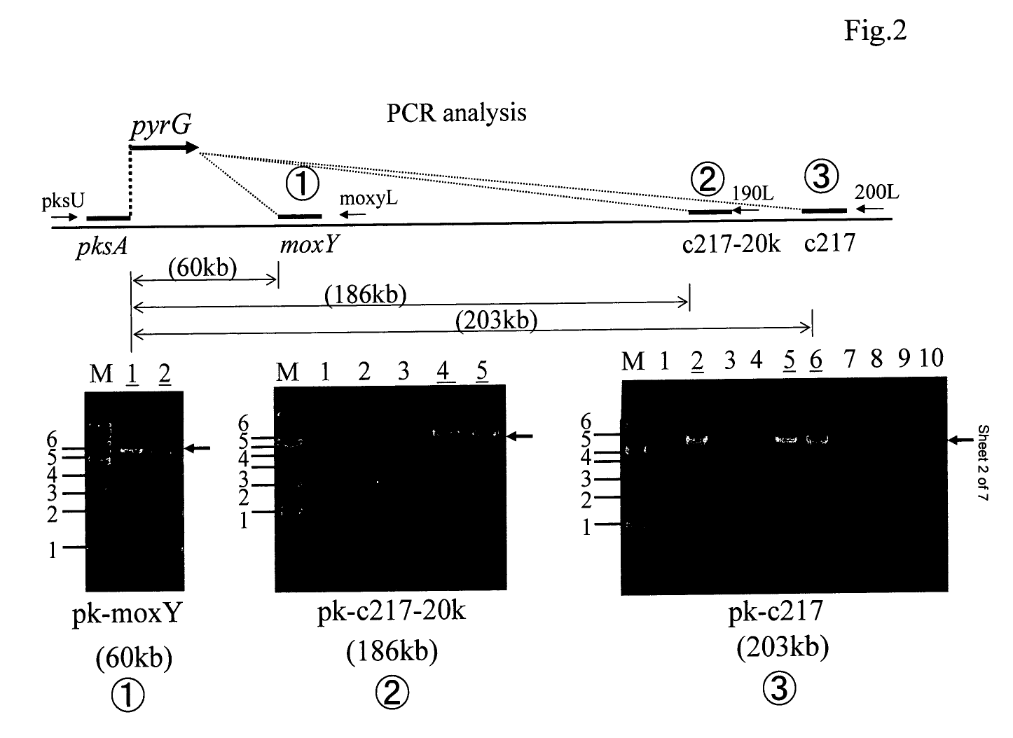

Aspergillus sojae ASKUPTR8 strain was then transformed with the vector (pPkvb1) for the deletion of a cluster of biosynthesis genes encoding Aflatoxin, the vector (pPk217-20k) for the deletion of a 190 kb region and the vector (pPk217) for the deletion of a 200 kb region, respectively. Genomic DNA was extracted from the resulting two transformants with the vector pPkvb1, the five transformants with the vector pPk217-20k and the ten transformants with the vector pPk217, and subjected to PCR and Southern hybridization for the purpose of confirmation of the integration of each vector. Thus, the PCR analysis revealed that a band was amplified with use of the primers pksU and AF-L (moxyL) amplified in the strains No. 1 and No. 2 having the deletion of the cluster of biosynthesis genes encoding Aflatoxin (FIG. 2, \textcircled{1} lanes 1 and 2). Similarly, it was revealed that a band was amplified with use of the primers pksU and 190L amplified in the strains No. 4 and No. 5 having the deletion of the 190 kb region (FIG. 2, \textcircled{2}, lanes 4 and 5), and that a band was amplified with use of the primers pksU and 200L amplified in the strains No. 5 and No. 6 having the deletion of the 200 kb region (FIG. 2, \textcircled{3}, lanes 2, 5 and 6). On the other hand, no band was amplified with the genomic DNA from the strains having no deletion. Accordingly, it was estimated that the transformants with the above vectors also included a heterocaryon comprising both a nucleus having the target deletion and an original nucleus.

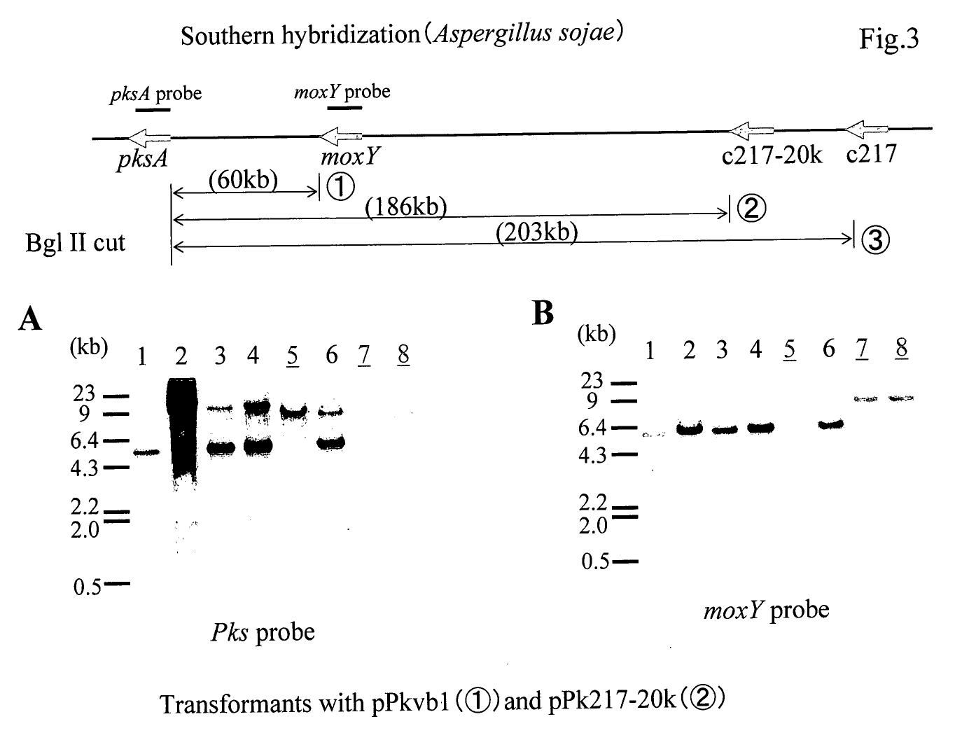

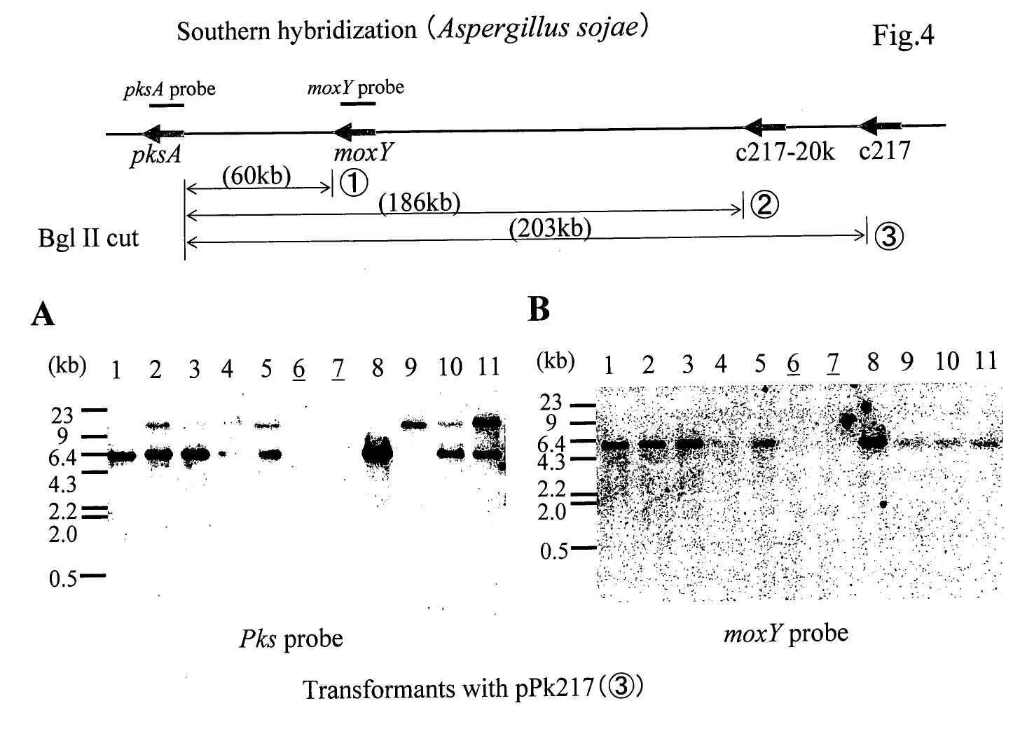

The Southern hybridization was carried out with genomic DNA of the transformants digested with BglII. It was observed that a band of 5.7 kb hybridized with the pksA probe in a parent strain (FIG. 3A, lane 1) was shifted to 10 kb in the strains No. 1 and No. 2 having the deletion of the cluster of biosynthesis genes encoding Aflatoxin (FIG. 3A, lanes 7, 8) and to 15 kb in the strain No. 4 having the deletion of the 190 kb region (FIG. 3A, lane 5). It was also observed that a band of 5.7 kb hybridized with the moxY probe in a parent strain (FIG. 3 B, lane 1) was shifted to 10 kb in the strains No. 1 and No. 2 having the deletion of the cluster of biosynthesis genes encoding Aflatoxin while it was not found in the strain having the deletion of a 190 kb region (FIG. 3B, lane 5). Similarly, a band of 5.7 kb hybridized with the pksA probe in a parent strain (FIG. 4A, lane 1) was shifted to 15 kb in the strains No. 6 and No. 7 having the deletion of the 200 kb region (FIG. 4A, lanes 6, 7). It was also observed that a band of 5.7 kb hybridized with the moxY probe in a parent strain (FIG. 4B, lane 1) was not found in the strains No. 6 and No. 7 having the deletion of a 200 kb region (FIG. 4B, lanes 6, 7). From these results, it was confirmed that the target region was finally deleted in the strains 1 and 2 having the deletion of the cluster of biosynthesis genes encoding Aflatoxin, the strain No. 4 having the deletion of the 190 kb region and the strains No. 6 and No. 7 having the deletion of the 200 kb region. The frequency of obtaining the desired transformants was 100% (2 out of 2 strains) in the strains having the deletion of the cluster of biosynthesis genes encoding Aflatoxin, 20% (one out of 5 strains) in the strains having the deletion of 190 kb region, and 20% (2 out of 10 strains) in the strains having the deletion of 200 kb region.

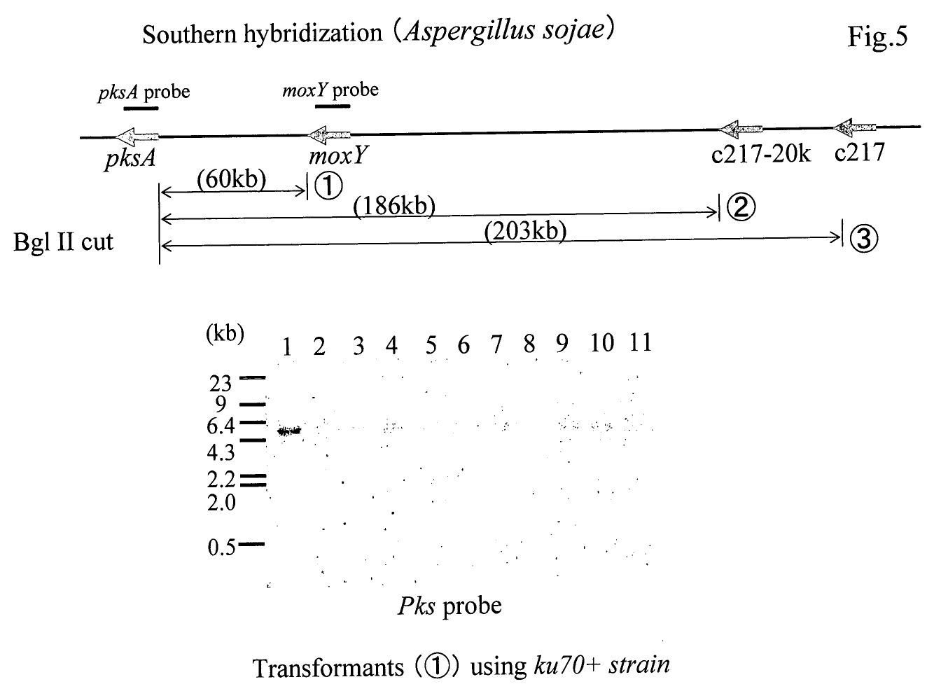

Next, preparation of a strain having the deletion of the cluster of biosynthesis genes encoding Aflatoxin was done using a strain wherein ku70 gene was not disrupted as a parent strain (A. sojae I-6) according to Non-Patent Document 1. Thus, said strain was transformed with the vector (pPkvb1) for the deletion of a cluster of biosynthesis genes encoding Aflatoxin. Genomic DNA was extracted from the resulting ten transformants and subjected to the Southern hybridization as shown in FIG. 5. There was no change in band pattern with the pksA probe in the parent strain (FIG. 5, lane 1) and the transformant (FIG. 5, lanes 2-11). Thus, the strain having the deletion of the cluster of biosynthesis genes encoding Aflatoxin could not be obtained by using the strain wherein ku70 gene was not disrupted.

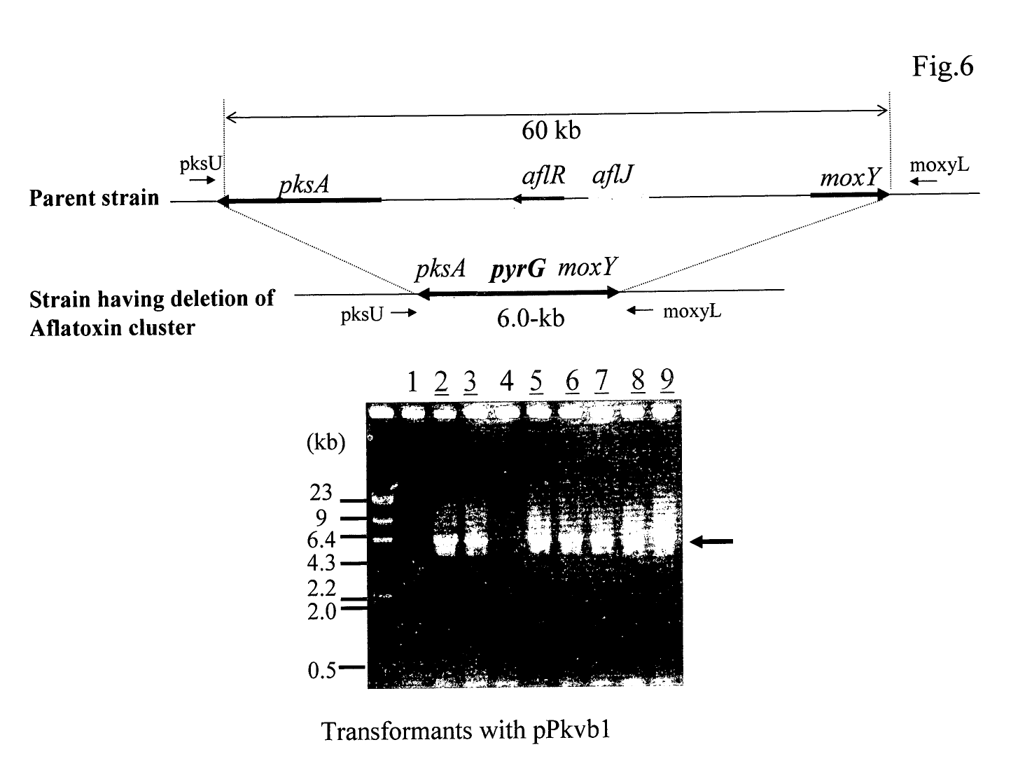

A. oryzae was similarly transformed with the vector pPkvb1 for the deletion of the cluster of biosynthesis genes encoding Aflatoxin. Genomic DNA was extracted from the resulting eight transformants obtained from A. oryzae RkuN16ptr1 (wh, ΔpyrG, Ku70::ptrA), and subjected to PCR using the primers pksU and moxyL. It was observed that a 6 kb band was amplified in 7 out of the 8 strains (FIG. 6, lanes 2, 3, 5-9) while no band was amplified in the parent strain. These results showed that there was at least one nucleus having the deletion of Aflatoxin cluster in the transformants wherein the 6 kb band was amplified.

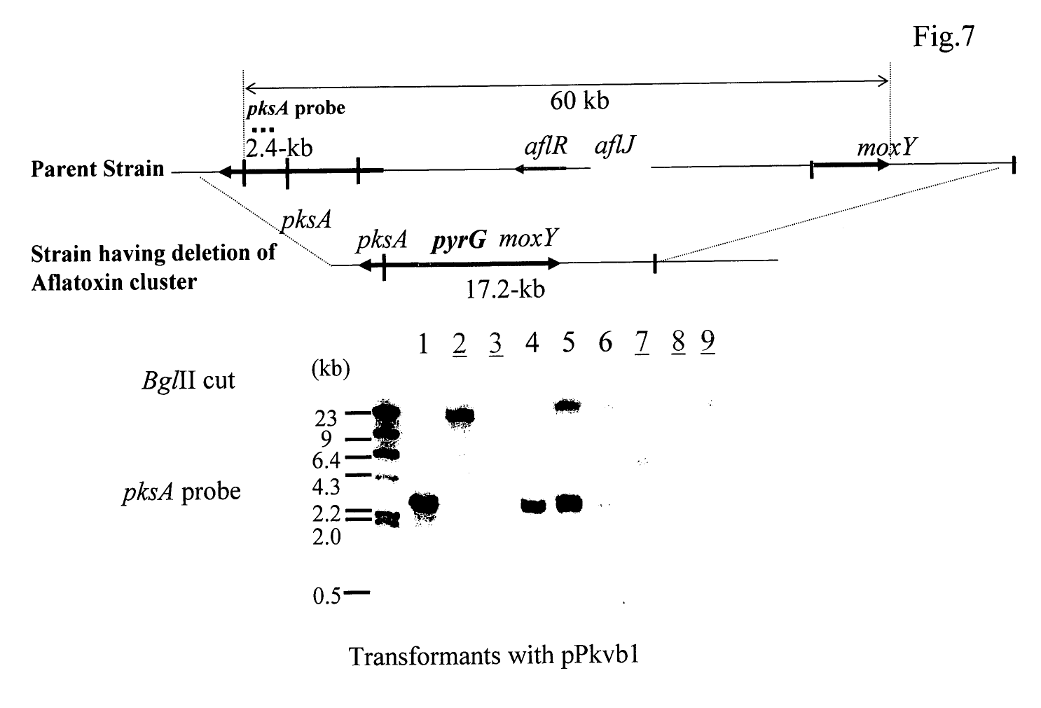

The Southern hybridization was carried out with genomic DNA of the transformants digested by Bgl II. It was observed that a band of 2.4 kb hybridized with the pksA probe in a parent strain (FIG. 7, lane 1) was shifted in 5 out of the 8 strains (FIG. 7, lanes 2, 3 7-9), showing the cluster of biosynthesis genes encoding Aflatoxin had been deleted in these transformants. As both 2.4 kb and 17.2 kb bands were observed in two out of 8 strains, these strains were confirmed to be a heterocaryon comprising both a nucleus having the deletion of the Aflatoxin cluster and an original nucleus (FIG. 7, lanes 5, 6).

A. oryzae RIB40 ΔpyrG strain wherein Ku70 was not disrupted was similarly transformed with said vector pPkvb1. The Southern hybridization analysis with respect to the resulting ten transformants revealed that only Bgl II band of 2.4 kb was observed in both each transformant and the parent strain, confirming that Aflatoxin genes had not been deleted.

The above frequencies were summarized in Table 2. It has been shown that a strain having the deletion of a large chromosomal region could be obtained with a high frequency by using the Ku70-disruption strains of A. oryzae and A. sojae while such strains having the deletion could not be obtained from a strain wherein Ku70 was not disrupted. These results show that a strain having the deletion of a large region such as that of at least about 200 kb, which has been very difficult to prepared, is now efficiently obtained by the present invention.

TABLE 2

| | Strain | | | | | |
|---|---|---|---|---|---|---|
| | A.sojae | | | | A. oryzae | |
| | Gene-type | | | | | |
| | Δku70 | | | ku70+ | Δku70 | ku70+ |
| | deleted region | | | | | |
| | 60 kb | 100 kb | 200 kb | 60 kb | 60 kb | 60 kb |
| Number of | 2 | 5 | 10 | 10 | 8 | 10 |
| transformants | | | | | | |
| Number of | 2 | 1 | 2 | 0 | 5 | 0 |
| transformant | | | | | | |
| having the dele- | | | | | | |
| tion | | | | | | |
| Frequencey (%) | 100 | 20 | 20 | 0 | 62.5 | 0 |

Additionally, preparation of a strain having the deletion of another large region was obtained, which region is not located in the vicinity of telomere of the chromosome No. 3 where the cluster of biosynthesis genes encoding Aflatoxin are present. Thus, deletion was tried with respect to a region of about 15 kb comprising a tannase gene in the vicinity of centromere of the chromosome No. 8 by using Aspergillus oryzae RkuN16ptr1 strain (wh, ΔpyrG, Ku70::ptrA). It was confirmed that the above deletion occurred in all of the resulting ten transformants. Accordingly it was shown that use of the Ku70-disruption strain enabled to efficiently obtain a strain having the deletion of a large region at any site in chromosome.

### INDUSTRIAL APPLICABILITY

The present invention makes it possible not only to efficiently produce Aspergillus strains having the deletion of a certain region that should be preferably removed for increase of safety of foods such as the cluster of biosynthesis genes encoding Aflatoxin, but also to modify Aspergillus strains at a chromosomal level so as to be of great advantage in industrial fields.

What is claimed is:

1) A method for the deletion of a large chromosomal region comprising transforming a transformant having an increased frequency of homologous recombination due to suppression of a Ku gene, which is a mitosporic filamentous fungus belonging to Trichocomaceae with a vector for the deletion of a chromosomal region, which comprises a pair of homologous region arms having a nucleotide sequence that is homologous with a fragment at either end of said chromosomal region, and deleting the chromosomal region by means of homologous recombination between the resulting transformant and said vector.

2) The method according to claim 1, wherein the transformant having an increased frequency of homologous recombination is Aspergillus sojae ASKUPTR8 strain (wh, ΔpyrG, Ku70::ptrA) (FERM BP-10453).

3) The method according to claim 1, wherein the transformant having an increased frequency of homologous recombination is Aspergillus oryzae RkuN16ptr1 strain.

4) The method according to claim 1, wherein a length of the homologous region arms is 1-3 kb.

5) The method according to any one of claim 4, wherein the homologous region arm has a nucleotide sequence of a gene or a part thereof that is located in the most marginal part at either end of the chromosomal region to be deleted.

6) The method according to claim 1, wherein a selective marker gene is comprised between the pair of the homologous region arms of the vector for the deletion of a chromosomal region.

7) The method according to claim 6, wherein the selective marker gene is selected from pyrG, sC or niaD.

8) The method according to claim 6, wherein a length of the chromosomal region to be deleted is 15-200 kb.

9) The method according to claim 8, wherein the chromosomal region to be deleted constitutes a cluster of biosynthesis genes encoding a toxic substance.

10) The method according to claim 8, wherein the toxic substance is Aflatoxin.

11) A transformant produced by the method according claim 1, which has the deletion of a part of chromosomal region.
